(19) United States
(12) Patent Application Publication
(10) Pub. No.: US 2020/0289693 A1
(43) Pub. Date: Sep. 17, 2020

(54) SYSTEM AND METHOD FOR DETECTING AND REMOVING ODOR AND BACTERIA FROM A SEALED VOLUME OF AN APPLIANCE USING OZONE

(71) Applicant: Haier US Appliance Solutions, Inc., Wilmington, DE (US)

(72) Inventors: Je Kwon Yoon, Seongnam (KR); JaeHyo Lee, Seoul (KR)

(21) Appl. No.: 16/351,638

(22) Filed: Mar. 13, 2019

(51) Int. Cl. A61L 9/04; F25D 17/04; A61L 9/12; D06F 35/00; A47L 15/42; D06F 58/28; A47L 15/00; F24C 15/20

(52) U.S. Cl. CPC A61L 9/046; A47J 37/0629; A61L 9/12; D06F 35/001; A47L 15/424; D06F 58/28; A47L 15/4276; A47L 15/0015; A47L 15/0036; F24C 15/2014; F25D 2317/0416; A61L 2209/212; A61L 2209/111; A61L 2209/13; F25D 17/042

(57) ABSTRACT

A method and system are provided that include features for operating an appliance in an odor removal cycle. In one aspect, an appliance has a housing defining a sealable volume and includes an ozone generator, an ozone detection device, and a controller. In an odor removal cycle, the controller causes the ozone generator to inject, at a predetermined injection interval, predefined dosages of ozone into the sealable volume of the appliance. The controller monitors the concentration level within the sealable volume after each injection based on inputs received from the ozone detection device. The controller ascertains when the concentration level reaches a maximum concentration level threshold, and when this occurs, the controller can cease the injections and can activate one or more ozone removal devices to remove the ozone from the sealable volume to a safe level.

### FIELD OF THE INVENTION

The subject matter of the present disclosure relates generally to a system and method for detecting and removing odor and bacteria from a sealable volume of an appliance using ozone.

### BACKGROUND OF THE INVENTION

Odor and bacteria within a sealed volume of an appliance can be unpleasant to consumers. Many different types of appliances include sealed volumes in which bacteria can grow and odor can emanate if left unaddressed. For instance, refrigerator appliances can include one or chilled chambers for storing food items. Storing food items for too long can cause mold and bacteria, including Psychrophilic bacteria, which can survive in a cold environment. To remove odors from the chilled chambers, consumers are typically directed to use baking soda. While odors can be removed by the baking soda technique, this technique is not able to remove any bacteria from the chilled chambers. Thus, the odors will likely return. Further, washing machines and dryers also include sealable volumes. To remove odors/bacteria therefrom, consumers are typically directed to run a full wash cycle or drying cycle. Running a full cycle to remove odor/bacteria can require significant time & energy. Dishwashers, air conditioners, and microwaves/ovens can also include sealable volumes. Dishwashers typically do not include odor removal systems, and in some instances, foul smelling odors can be absorbed by the gaskets and plastic components thereof. For air conditioners, evaporators can smell bad if not operated for a while and moisture is not fully removed. For microwaves or ovens, constantly heating various types of food items can generator odor into various parts of the microwave/oven (fan, rotation plate, etc.).

Ozone can be an effective sterilant and oxidizer for removing odors, bacteria, and viruses in a sealable volume. Ozone generators can be used to inject ozone into a sealable volume. However, there are currently no satisfactory systems or methods to ensure that the amount of ozone within the sealable volume does not exceed unsafe levels. Exposure of ozone must be avoided by consumers as high concentration levels of ozone may harm consumers' respiratory systems. Accordingly, when ozone is used to deodorize or remove bacteria from a sealable volume, consumers are instructed to leave the area and to return only after the ozone is reverted to oxygen. This can be an inconvenience to users and current systems can be ineffective in actually removing the odor/bacteria from the sealed volume. Furthermore, in some instances, conventional systems can inject too little ozone into the sealed volume. In such instances, the ozone injection is ineffective in removal of bacteria/odor from the sealed volume.

Accordingly, a system and method that address one or more of the challenges noted above would be useful.

### BRIEF DESCRIPTION OF THE INVENTION

Aspects and advantages of the invention will be set forth in part in the following description, or may be apparent from the description, or may be learned through practice of the invention.

In one aspect, an appliance is provided. The appliance includes a housing defining a sealable volume. The appliance also includes an ozone generator operable to dispense ozone into the sealable volume. Further, the appliance includes an ozone detection device operable to detect a concentration level of ozone within the sealable volume. In addition, the appliance includes a controller communicatively coupled with the ozone generator and the ozone detection device. The controller is configured to: i) cause, at a predetermined injection interval, the ozone generator to inject a predefined dosage of ozone into the sealable volume; ii) receive, from the ozone detection device, an input indicative of the concentration level of ozone within the sealable volume; iii) determine the concentration level of ozone within the sealable volume based at least in part on the received input; and iv) ascertain whether the determined concentration level has reached a maximum concentration level threshold. Further, the controller iteratively i) causes, ii) receives, iii) determines, and iv) ascertains until the determined concentration level reaches the maximum concentration level threshold or a maximum generator on time has elapsed.

In another aspect, a method for operating an appliance in an odor removal cycle is provided. The method includes injecting, at a predetermined injection interval, a predefined dosage of ozone into a sealable volume of the appliance. Further, the method includes measuring, after each injection of the predefined dosage of ozone into the sealable volume of the appliance, a concentration level of ozone within the sealable volume. In addition, the method includes ascertaining whether the concentration level has reached a maximum concentration level threshold, and wherein if the concentration level has reached the maximum concentration level threshold, then no further injections of the predefined dosage of ozone are made.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE DRAWINGS

A full and enabling disclosure of the present invention, including the best mode thereof, directed to one of ordinary skill in the art, is set forth in the specification, which makes reference to the appended figures, in which:

FIG. 1 provides a perspective view of a refrigerator appliance according to example embodiments of the present subject matter;

FIG. 2 provides a perspective view of the refrigerator appliance of FIG. 1, wherein refrigerator doors of the refrigerator appliance are depicted in an open position to reveal a fresh food chamber of the refrigerator appliance;

FIG. 3 provides a schematic view of an example appliance equipped with an ozone monitoring system according to example embodiments of the present subject matter;

FIG. 4 provides a flow diagram of a method for operating an appliance in an odor removal cycle according to example embodiments of the present subject matter; and

FIGS. 5, 6, and 7 provide graphs depicting a concentration level of ozone as a function of time for three different scenarios according to example embodiments of the present subject matter.

### DETAILED DESCRIPTION OF THE INVENTION

Reference now will be made in detail to embodiments of the invention, one or more examples of which are illustrated in the drawings. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment can be used with another embodiment to yield a still further embodiment. Thus, it is intended that the present invention covers such modifications and variations as come within the scope of the appended claims and their equivalents. As used herein, terms of approximation, such as “approximately,” “substantially,” or “about,” refer to being within a fifteen percent (15%) margin of error.

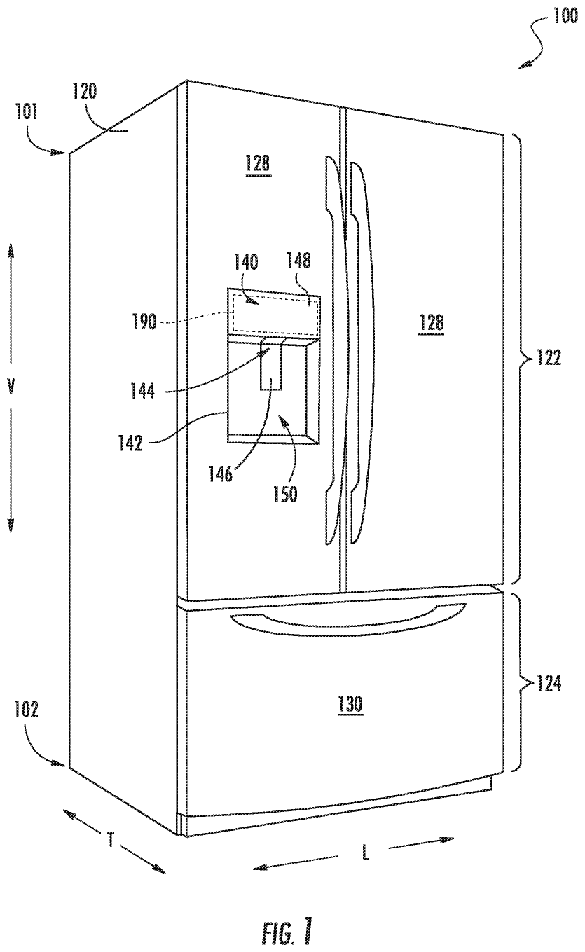

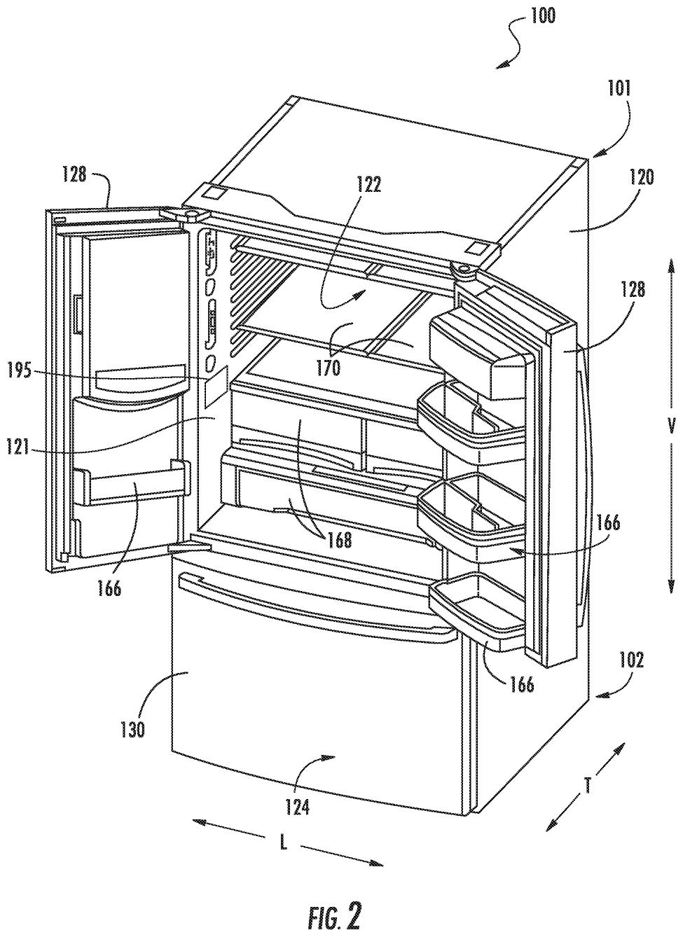

FIGS. 1 and 2 provide various views of a refrigerator appliance 100 according to example embodiments of the present subject matter. Particularly, FIG. 1 provides a perspective view of refrigerator appliance 100 and FIG. 2 provides a perspective view of refrigerator appliance 100 having multiple refrigerator doors 128 in the open position. As shown, refrigerator appliance 100 includes a cabinet or cabinet 120 that extends between a top 101 and a bottom 102 along a vertical direction V. Cabinet 120 also extends along a lateral direction L and a transverse direction T, each of the vertical direction V, lateral direction L, and transverse direction T being mutually perpendicular to one another. In turn, vertical direction V, lateral direction L, and transverse direction T define an orthogonal direction system.

Cabinet 120 includes a liner 121 that defines one or more sealable volumes. For this embodiment, the sealable volumes are chilled chambers configured for receipt of food items for storage. In particular, liner 121 defines a fresh food chamber 122 positioned at or adjacent top 101 of cabinet 120 and a freezer chamber 124 arranged at or adjacent bottom 102 of cabinet 120. As such, refrigerator appliance 100 is generally referred to as a bottom mount refrigerator. It is recognized, however, that the benefits of the present disclosure apply to other types and styles of appliances such as, e.g., a top mount refrigerator appliance, a side-by-side style refrigerator appliance, or a range appliance. Further, as will be explained herein, the benefits of the present disclosure apply to other types of appliances as well. Consequently, the description set forth herein is for illustrative purposes only and is not intended to be limiting in any aspect to any particular refrigerator chamber configuration.

Refrigerator doors 128 are rotatably hinged to an edge of cabinet 120 for selectively accessing fresh food chamber 122. In addition, a freezer door 130 is arranged below refrigerator doors 128 for selectively accessing freezer chamber 124. Freezer door 130 is attached to a freezer drawer (not shown) slidably mounted within freezer chamber 124. Refrigerator doors 128 and freezer door 130 are shown in the closed configuration in FIG. 1.

In some embodiments, refrigerator appliance 100 also includes a dispensing assembly 140 for dispensing liquid water and/or ice. Dispensing assembly 140 includes a dispenser 142 positioned on or mounted to an exterior portion of refrigerator appliance 100, e.g., on one of refrigerator doors 128. Dispenser 142 includes a discharging outlet 144 for accessing ice and liquid water. An actuating mechanism 146, shown as a paddle, is mounted below discharging outlet 144 for operating dispenser 142. In alternative exemplary embodiments, any suitable actuating mechanism may be used to operate dispenser 142. For example, dispenser 142 can include a sensor (such as an ultrasonic sensor) or a button rather than the paddle. A user interface panel 148 is provided for controlling the mode of operation. For example, user interface panel 148 includes a plurality of user inputs (not labeled), such as a water dispensing button and an ice-dispensing button (e.g., for selecting a desired mode of operation such as crushed or non-crushed ice).

Discharging outlet 144 and actuating mechanism 146 are an external part of dispenser 142 and are mounted in a dispenser recess 150. Dispenser recess 150 is positioned at a predetermined elevation convenient for a user to access ice or water and enabling the user to access ice without the need to bend-over and without the need to open refrigerator doors 128.

According to the illustrated embodiment, various storage components are mounted within fresh food chamber 122 to facilitate storage of food items therein as will be understood by those skilled in the art. In particular, the storage components include storage bins 166, drawers 168, and shelves 170 that are mounted within fresh food chamber 122. Storage bins 166, drawers 168, and shelves 170 are configured for receipt of food items (e.g., beverages and/or solid food items) and may assist with organizing such food items. As an example, drawers 168 can receive fresh food items (e.g., vegetables, fruits, and/or cheeses) and increase the useful life of such fresh food items.

Operation of the refrigerator appliance 100 can be controlled or regulated by a controller 190. As will be described in detail below, controller 190 may include multiple modes of operation or sequences that control or regulate various portions of refrigerator appliance 100 according to one or more discrete criteria.

In some embodiments, controller 190 is operably coupled to user interface panel 148 and/or various other components, as will be described below. User interface panel 148 provides selections for user manipulation of the operation of refrigerator appliance 100. As an example, user interface panel 148 may provide for selections between whole or crushed ice, chilled water, and/or specific modes of operation. In response to one or more input signals (e.g., from user manipulation of user interface panel 148 and/or one or more sensor signals), controller 190 may operate various components of the refrigerator appliance 100.

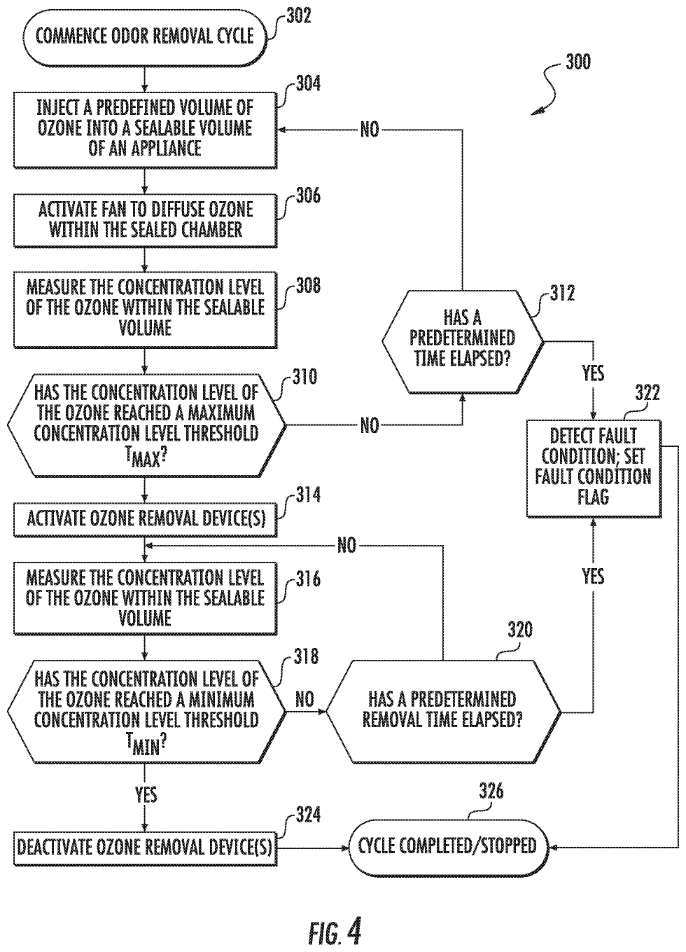

Controller 190 may include a memory and one or more microprocessors, CPUs or the like, such as general or special purpose microprocessors operable to execute programming instructions or micro-control code associated with operation of refrigerator appliance 100. The memory may represent random access memory such as DRAM, or read only memory such as ROM or FLASH. In some embodiments, the processor executes non-transitory programming instructions stored in memory. For certain embodiments, the instructions include a software package configured to operate appliance 100 and, e.g., execute an operation routine including the example method (300) described below with reference to FIG. 4. The memory may be a separate component from the processor or may be included onboard within the processor. Alternatively, controller 190 may be constructed without using a microprocessor, e.g., using a combination of discrete analog and/or digital logic circuitry (such as switches, amplifiers, integrators, comparators, flip-flops, AND gates, and the like) to perform control functionality instead of relying upon software.

Controller 190, or portions thereof, may be positioned in a variety of locations throughout refrigerator appliance 100. In example embodiments, controller 190 is located within the user interface panel 148 as shown in FIG. 1. In other embodiments, the controller 190 may be positioned at any suitable location within refrigerator appliance 100, such as for example within a fresh food chamber, a freezer door, etc. In additional or alternative embodiments, controller 190 is formed from multiple components mounted at discrete locations within or on refrigerator appliance 100. Input/output (“I/O”) signals may be routed between controller 190 and various operational components of refrigerator appliance 100. For example, user interface panel 148 may be operably coupled (e.g., directly or indirectly electrically coupled) to controller 190 via one or more signal lines or shared communication busses.

In addition, as shown in FIG. 2, refrigerator appliance 100 can include an ozone monitoring system, as represented by 195. Ozone monitoring system 195 is operable to detect odor/bacteria/viruses in a sealed (or air tight) area of refrigerator appliance 100, such as e.g., fresh food chamber 122 and/or freezer chamber 124. Various components of ozone monitoring system 195 can be communicatively coupled with and controlled by controller 190. An example monitoring system for an appliance is provided below.

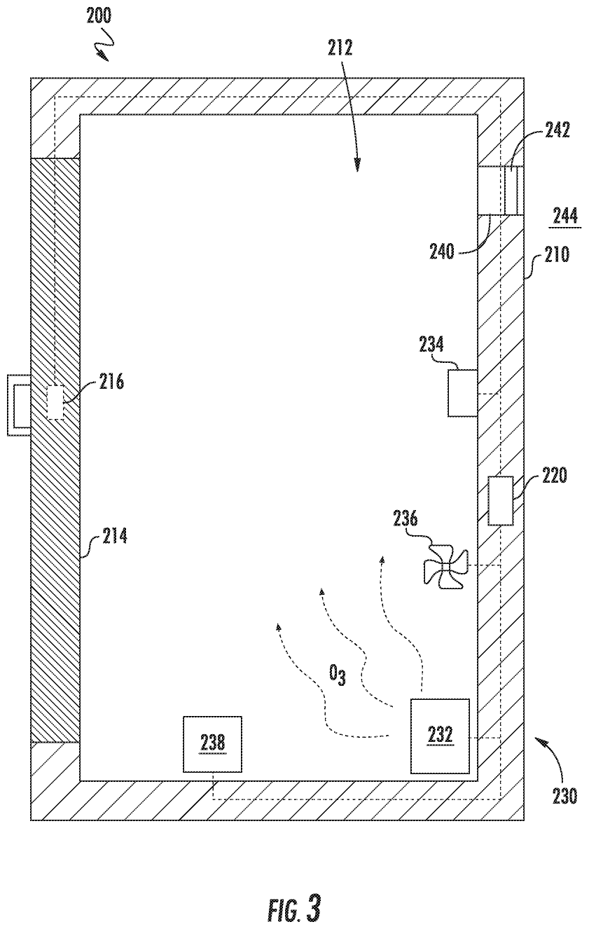

FIG. 3 provides a schematic view of an example appliance 200 equipped with an ozone monitoring system 230 according to example embodiments of the present subject matter. For instance, appliance 200 of FIG. 3 can be the refrigerator appliance 100 of FIGS. 1 and 2 and ozone monitoring system 230 can be ozone monitoring system 195 depicted in FIG. 2. However, the appliance 200 of FIG. 3 can be any suitable appliance having a sealable volume and the ozone monitoring features described below. By way of example, without limitation, appliance 200 of FIG. 3 can be a washing machine appliance, a dryer appliance, a microwave appliance, an oven appliance, or an air conditioner appliance. In addition, compliance 200 of FIG. 3 can be a refrigerator appliance having a different configuration than refrigerator appliance 100 of FIGS. 1 and 2.

As depicted in FIG. 3, appliance 200 includes a housing 210 defining a sealable volume 212. For example, the housing 210 can be cabinet 120 of refrigerator appliance 100 and sealable volume 212 can be one of the chilled chambers 122, 124 thereof. A door 214 is operatively coupled with housing 210 for providing selective access to the sealable volume 212. Door 214 is movable between a closed position in which sealable volume 212 is sealed and an open position. In some embodiments, in the closed position, door 214 hermetically seals sealable volume 212 such that sealable volume 212 is a sealed volume. In the open position, door 214 does not hermetically seal sealable volume 212; thus, when door 214 is in the open position, sealable volume 212 is not sealed. For this embodiment, door 214 includes a door lock 216 for selectively locking door 214, e.g., in the closed position. As will be explained herein, during an odor removal cycle, a controller 220 of appliance 200 can cause door lock 216 to keep or maintain door 214 in the closed position, e.g., until completion of the cycle.

Appliance 200 also includes ozone monitoring system 230. Generally, ozone monitoring system 230 is operable to remove bacteria and odor from sealable volume 212 in a safe and efficient manner. Ozone monitoring system 230 includes an ozone generator 232 operable to dispense or inject ozone into the sealable volume 212. For instance, ozone generator 232 is depicted in FIG. 3 injecting ozone O₃ into sealable volume 212. If odor, bacteria, and/or viruses are present within sealable volume 212, the ozone O₃ injected therein can be “consumed” or reverted to oxygen molecules after destroying odor, bacteria, and/or viruses. Ozone O₃ is one suitable sterilant and oxidizer effective in destroying odor, bacteria and viruses in sealable volume 212. In some alternative embodiments, generator device 232 can generate another sterilant/oxidizer, such as e.g., a suitable variant of ozone O₃.

Ozone monitoring system 230 also includes an ozone detection device 234. Ozone detection device 234 can be any suitable sensor operable to sense or detect the ozone concentration within sealable volume 212. For instance, after ozone generator 232 injects a predefined dosage of ozone into sealable volume 212, ozone detection device 234 can sense the ozone concentration within sealable volume 212. One or more signals indicative of the concentration level of ozone within sealable volume 212 can be routed from ozone detection device 234 to controller 220 for processing, which as shown in FIG. 3, is communicatively coupled thereto.

In some example embodiments, optionally, ozone monitoring system 230 includes an air handler 236 (e.g., a fan). Air handler 236 is operable to facilitate diffusion of ozone O₃ within sealable volume 212. For instance, prior to, simultaneously with, or after ozone generator 232 injects ozone O₃ into sealable volume 212, controller 220 can activate air handler 236 to move air about sealable volume 212. Consequently, air handler 236 assists with mixing of ozone O₃ with the existing air within sealable volume 212. This can, for example, cause more rapid diffusion of ozone O₃ with the existing air within sealable volume 212. Controller 220 can also deactivate air handler 236, e.g., at the end of the odor removal cycle.

Further, in some example embodiments, optionally, ozone monitoring system 230 includes an ozone destructor device 238. Ozone destructor device 238 is operable to reduce the concentration level of ozone within the sealable volume 212. For instance, ozone O₃ within sealable volume 212 can be destructed by ozone destructor device 238 via a catalyst, such as e.g., manganese dioxide MnO₂. Ozone destructor device 238 can destruct ozone O₃ within sealable volume 212 at any suitable time. For instance, as will be explained in detail herein, controller 220 can cause ozone destructor device 238 to destruct or reduce the concentration level of ozone O₃ when the ozone concentration level within sealable volume 212 reaches a threshold. In addition, in some embodiments, ozone destructor device 238 can impart heat into the sealable volume 212. In this way, ozone O₃ within sealable volume 212 can be destructed.

Controller 220 of appliance 200 is also a component of system 230. In some embodiments, controller 220 of system 230 can be dedicated solely to performing operations for operating appliance 200 in an odor removal cycle. In yet other embodiments, in addition to performing operations for operating appliance 200 in an odor removal cycle, controller 220 can perform other operations associated with appliance 200. Controller 220 can be configured the same or similar to the controller 190 of refrigerator appliance 100 of FIGS. 1 and 2. Particularly, controller 220 can include one or more memory devices and one or more processing devices. For instance, the processing devices can be microprocessors, CPUs, or the like, such as general or special purpose microprocessors operable to execute programming instructions or micro-control code associated with operations of appliance 200. The memory devices can include random access memory such as DRAM, and/or read only memory such as ROM or FLASH. In some embodiments, the one or more processing devices execute non-transitory programming instructions stored in the one or more memory devices. For certain embodiments, the instructions include a software package configured to operate appliance 200, e.g., in an odor removal cycle. The one or memory devices can be separate components from the one or more processors or may be included onboard with the processors. Alternatively, controller 220 can be constructed without using a microprocessor, e.g., using a combination of discrete analog and/or digital logic circuitry (such as switches, amplifiers, integrators, comparators, flip-flops, AND gates, and the like) to perform control functionality instead of relying upon software.

Controller 220 can send and receive signals from various components of appliance 200, and particularly, components of ozone monitoring system 230. As depicted in FIG. 3, controller 220 is communicatively coupled with ozone generator 232, ozone detection device 234, air handler 236, ozone destructor device 238, and door lock 216. Controller 220 can also be communicatively coupled with other components of appliance 200 as well. Controller 220 can be communicatively coupled with these various devices in any suitable manner, e.g., a suitable wired or wireless communication link. Controller 220 can control appliance 200 in an odor removal cycle in a manner described below as set forth in method (300).

FIG. 4 provides a flow diagram of a method (300) for operating an appliance in an odor removal cycle according to example embodiments of the present subject matter. For instance, the method (300) can be implemented to operate appliance 200 of FIG. 3 in an odor removal cycle. Appliance 200 can be any suitable type of appliance, including, without limitation, a refrigerator appliance, a washing machine appliance, a dryer appliance, a microwave appliance, an oven appliance, or an air conditioner appliance. Reference numerals used to denote certain features of appliance 200 of FIG. 3 will be utilized below to provide context to method (300). In addition, it will be appreciated that method (300) can be modified, adapted, expanded, rearranged and/or omitted in various ways without deviating from the scope of the present subject matter.

At (302), the method (300) includes commencing an odor removal cycle. The odor removal cycle can be commenced in a number of suitable ways. For instance, a user can manually commence the odor removal cycle. For example, a user can manipulate one or more controls of the user interface of appliance 200. As another example, a user can start the odor removal cycle by utilizing an application on a remote user device communicatively coupled with controller 220 of appliance 200. Another suitable manner for commencing the odor removal cycle can include commencing the odor removal cycle at a predetermined interval, such as e.g., every week, every month, etc. In this manner, the odor removal cycle can be performed without user interaction with appliance 200.

At (304), the method (300) includes injecting, at a predetermined interval, a predefined dosage of ozone into a sealable volume of an appliance. For instance, with reference to FIG. 3, controller 220 can cause ozone generator 232 to inject a predefined dosage of ozone O₃ into sealable volume 212. The predefined dosage of ozone can be a known volume of ozone O₃. Accordingly, when a predefined dosage of ozone O₃ is injected into sealable volume 212, controller 220 can track the amount or volume of ozone O₃ dispensed or injected into sealable volume 212. As will be explained in further detail below, controller 220 can iteratively cause ozone generator 232 to inject a predefined dosage of ozone O₃ into sealable volume 212 at a predefined time interval. The predetermined time interval can be set based at least in part on the dosage amount and the volume of the sealable volume 212, among other possible criteria.

At (306), optionally, the method (300) includes activating an air handler to facilitate diffusion of the ozone within the sealable volume. For instance, the air handler can be air handler 236 of FIG. 4. Prior to, simultaneously with, or after ozone generator 232 injects ozone O₃ into sealable volume 212 at (304), controller 220 can activate air handler 236 to move air about sealable volume 212. As a result, injected ozone O₃ can be more rapidly mixed with the existing air within sealable volume 212. As noted previously, this can cause more rapid diffusion of ozone O₃ with the existing air within sealable volume 212. Air handler 236 can be activated with each injected dosage and can run for a predetermined time, can be activated after the first dosage and can run for the entire odor removal cycle, or can be activated until the occurrence of some event, such as e.g., when the concentration level of ozone O₃ within sealable volume 212 reaches a maximum concentration level threshold, among other possibilities.

At (308), the method (300) includes measuring a concentration level of ozone within the sealable volume after each injection of the predefined dosage of ozone into the sealable volume of the appliance. In some implementations, measuring the concentration level of ozone within the sealable volume includes receiving, from an ozone detection device, an input indicative of the concentration level of ozone within the sealable volume and then determining the concentration level of ozone within the sealable volume based at least in part on the received input.

For instance, with reference to FIG. 3, controller 220 can receive, from ozone detection device 234, an input indicative of a concentration level of ozone O₃ within the sealable volume 212. For example, controller 220 can receive one or more electrical signals indicative of the concentration level ozone O₃ within the sealable volume 212. Controller 220 can receive such signals, or the input, and can determine the concentration level of ozone O₃ within sealable volume 212 based at least in part on the received input. Controller 220 can determine the concentration level of ozone O₃ within sealable volume 212 in any suitable units, such as e.g., parts per million (ppm). Controller 220 can measure or determine the concentration level of ozone O₃ within sealable volume 212 at a predetermined diffusion time after each predefined dosage of ozone O₃. For instance, controller 220 can measure the concentration level of ozone O₃ within sealable volume 212 twenty (20) seconds (i.e., the predetermined diffusion time) after each predefined dosage of ozone O₃ is injected into sealable volume 212.

At (310), the method (300) includes ascertaining whether the concentration level has reached a maximum concentration level threshold. If the concentration level has reached the maximum concentration level threshold, then no further injections of the predefined dosage of ozone are made. For instance, referring to FIG. 3, controller 220 can ascertain whether the determined concentration level has reached the maximum concentration level threshold. Notably, controller 220 iteratively i) causes ozone generator 232 to inject a predefined dosage of ozone O₃ into sealable volume 212, ii) receives an input indicative of a concentration level of ozone O₃ within the sealable volume 212, iii) determines the concentration level of ozone O₃ within sealable volume 212 based at least in part on the received input, and iv) ascertains whether the concentration level has reached a maximum concentration level threshold at a predetermined time interval until the determined concentration level reaches the maximum concentration level threshold as determined at (310) or a maximum generator on time has elapsed as determined at (312).

At (312), if the concentration level has not reached the maximum concentration level threshold T_{MAX}, then the method (300) includes determining whether a maximum generator on time has elapsed. For instance, controller 220 can maintain a timer or clock. The timer can be started when the first ozone dosage is injected into sealable volume 212 at (304) and can terminate at the end of the maximum generator on time. In this way, ozone generator 232 is prevented from running indefinitely in the event of a failure condition. If the maximum generator on time has not elapsed as determined at (312), then method (300) reverts to (304) so that another predefined dosage of ozone can be injected into sealable volume 212 by ozone generator 232. If, however, the maximum generator on time has elapsed as determined at (312), then the method (300) proceeds to (322) where controller 220 determines that a fault condition is detected and can set a flag indicating the fault detected.

FIGS. 5, 6, and 7 present three (3) example scenarios in which method (300) may proceed through (304) through (312). Particularly, FIGS. 5, 6, and 7 provide graphs depicting a concentration level of ozone as a function of time for three different scenarios according to example embodiments of the present subject matter.

With reference to FIG. 5, in a first scenario, there may be negligible or no odor, bacteria, viruses, and/or other contaminants to remove from sealable volume 212. In such instances, injected ozone O₃ will not be “consumed,” and thus the concentration level of ozone O₃ will accumulate with each injected predefined dosage of ozone O₃. By way of example, as shown in FIG. 5, a number of ozone dosages are injected into sealable volume 212, including a first dosage D1, a second dosage D2, a third dosage D3, a fourth dosage D4, and a fifth dosage D5. The ozone dosages D1, D2, D3, D4, D5 are injected at a predetermined injection interval, as represented by I.

Notably, after the first dosage D1 of ozone O₃ is injected at (304) of method (300), the concentration level of ozone O₃ remains relatively constant for a time (e.g., until the second dosage D2 is injected). This represents that the injected ozone O₃ is not being “consumed” or reacting with odors, bacteria, viruses, and/or other contaminants within sealable volume 212. After the first dosage D1, controller 220 measures the concentration level of ozone O₃ within the sealable volume 212 at (308) of method (300), (e.g., controller 220 receives an input indicative of the concentration level from ozone detection device 234 and determines the concentration level based at least in part on the received input), and ascertains at (310) that the concentration level has not reached the maximum concentration level threshold T_{MAX}. Accordingly, the method (300) reverts to (304) if the maximum generator on time has not elapsed as determined at (312).

If the concentration level has not reached the maximum concentration level threshold T_{MAX} and the maximum generator on time has not elapsed, then at (304) controller 220 once again causes ozone generator 232 to inject a predefined dosage of ozone into sealable volume 212. For instance, as shown in FIG. 5, the second dosage D2 is injected by ozone generator 232. After the second dosage D2 of ozone O₃ is injected at (304), the concentration level of ozone O₃ remains relatively constant for a time (e.g., until the third dosage D3 is injected). This represents that the injected ozone O₃ is still not being “consumed” or reacting with odors, bacteria, viruses, and/or other contaminants within sealable volume 212. After the second dosage D2, controller 220 measures the concentration level of ozone O₃ within the sealable volume 212 at (308) of method (300), and ascertains at (310) that the concentration level has not reached the maximum concentration level threshold T_{MAX}, e.g., as shown in FIG. 5. Accordingly, the method (300) reverts to (304) if the maximum generator on time has not elapsed as determined at (312). This process continues until the determined concentration level reaches the maximum concentration level threshold T_{MAX} (e.g., at time t_{X} as shown in FIG. 5) or if the maximum generator on time has elapsed. If either of these conditions are met, then controller 220 ceases causing ozone generator 232 to inject ozone into sealable volume 212.

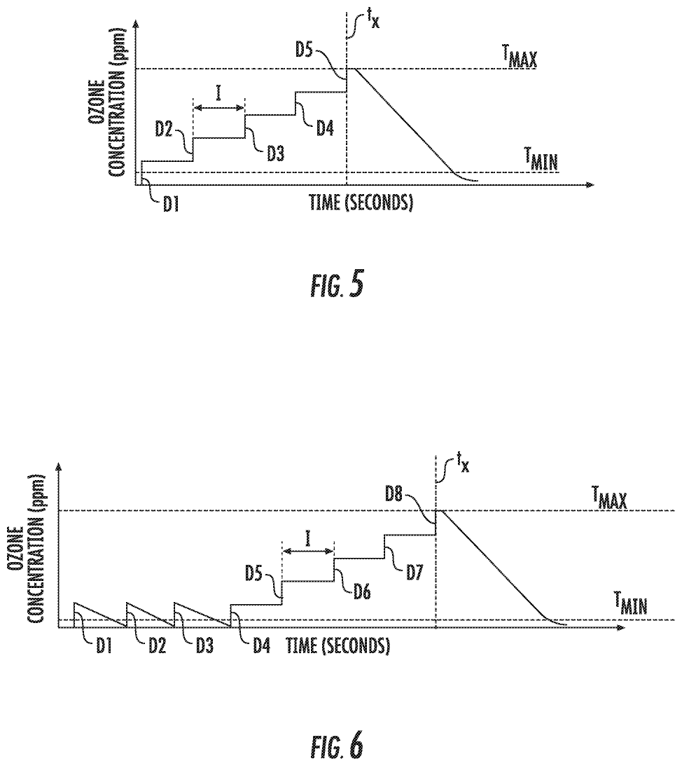

With reference to FIG. 6, in a second scenario, odor, bacteria, viruses, and/or some other contaminants exist in sealable volume 212 and can be removed with ozone O₃. By way of example, as shown in FIG. 6, a number of ozone dosages are injected into sealable volume 212, including a first dosage D1, a second dosage D2, a third dosage D3, a fourth dosage D4, and fifth dosage D5, a sixth dosage D6, a seventh dosage D7, and an eighth dosage D8. The ozone dosages D1, D2, D3, D4, D5, D6, D7, D8 are injected at a predetermined injection interval, as represented by I. The degree of odor/bacteria is measured based on the amount of time it takes to remove all odor/bacteria. If the ozone concentration level is increasing, it is an indication that all odor/bacteria is removed.

After the first dosage D1 of ozone O₃ is injected into sealable volume 212 at (304), the concentration level of ozone O₃ decreases (e.g., until the second dosage D2 is injected). This represents that the injected ozone O₃ is being “consumed” or reacting with odors, bacteria, viruses, and/or other contaminants within sealable volume 212. After the first dosage D1, controller 220 measures the concentration level of ozone O₃ within the sealable volume 212 at (308) of method (300), and ascertains at (310) that the concentration level has not reached the maximum concentration level threshold T_{MAX}. Accordingly, the method (300) reverts to (304) if the maximum generator on time has not elapsed as determined at (312). This process continues until the determined concentration level reaches the maximum concentration level threshold T_{MAX} as determined at (310) or if the maximum generator on time has elapsed as determined at (312).

After the method (300) loops through (304) through (312) for second and third dosages D2 and D3, after the fourth dosage D4 of ozone O₃ is injected at (304) of method (300), the concentration level of ozone O₃ remains relatively constant for a time (e.g., until the fifth dosage D5 is injected). This represents that the injected ozone O₃ is no longer being “consumed” or reacting with odors, bacteria, viruses, and/or other contaminants within sealable volume 212. With no further odors, bacteria, viruses, and/or other contaminants within sealable volume 212 for ozone O₃ to react with, the concentration level continues to increase in a stepwise function for dosages D5, D6, D7, and D8 until the concentration level of ozone O₃ reaches the maximum concentration level threshold T_{MAX} (e.g., at time t_{X} as shown in FIG. 6).

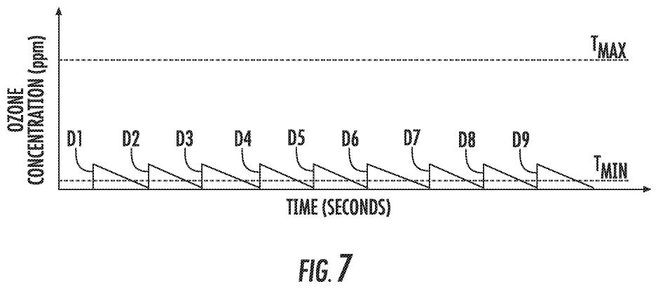

With reference to FIG. 7, in a third scenario, dosages of ozone O₃ are injected into sealable volume 212 at a predetermined injection interval I, yet the concentration level of ozone O₃ does not reach the maximum concentration level threshold T_{MAX} as determined at (310) before the maximum generator on time has elapsed as determined at (312). By way of example, as shown in FIG. 7, a number of ozone dosages are injected into sealable volume 212, including a first dosage D1, a second dosage D2, a third dosage D3, a fourth dosage D4, and fifth dosage D5, a sixth dosage D6, a seventh dosage D7, an eighth dosage D8, and a ninth dosage D9. The ozone dosages D1, D2, D3, D4, D5, D6, D7, D8, D9 are injected at the predetermined injection interval I, as noted above.

As shown, after each dosage of ozone O₃ injected into sealable volume 212 by ozone generator 232, the concentration level decreases (e.g., until a subsequent dosage is injected). This represents that the injected ozone O₃ is being “consumed” or reacting with odors, bacteria, viruses, and/or other contaminants within sealable volume 212. Thus, method (300) continues within the (304) to (312) loop until the determined concentration level reaches the maximum concentration level threshold T_{MAX} as determined at (310) or if the maximum generator on time has elapsed as determined at (312). In the third scenario shown in FIG. 7, the determined concentration level does not reach the maximum concentration level threshold T_{MAX} before the maximum generator on time elapses. Thus, as shown in FIG. 4, the method (300) proceeds to (322).

At (314), in some implementations, the method (300) includes activating one or more ozone removal devices. For instance, in some implementations, activating the one or more ozone removal devices includes activating ozone destructor device 238 to reduce the concentration level of ozone O₃ within sealable volume 212. For instance, if the determined concentration level reaches the maximum concentration level threshold T_{MAX} as determined at (310), controller 220 is configured to activate ozone destructor device 238 to reduce the concentration level of ozone within sealable volume 212. For instance, ozone destructor device 238 can reduce the concentration level of ozone O₃ within sealable volume 212 via a catalyst, such as e.g., manganese dioxide MnO₂. Ozone destructor device 238 can destruct ozone O₃ within sealable volume 212 when the ozone concentration level within sealable volume 212 reaches the maximum concentration level threshold T_{MAX}, e.g., as shown in FIG. 5 after the fifth dosage D5 and in FIG. 6 after the eighth dosage D8. In yet other implementations of method (300), the ozone destructor device 238 can reduce the concentration level of ozone O₃ within sealable volume 212 by imparting heat to sealable volume 212.

In some implementations, activating the one or more ozone removal devices at (314) includes causing a damper to move to an open position such that ozone can be exhausted from sealable volume. In such implementations, when the damper is moved to the open position, the ozone O₃ within sealable volume 212 can be passively exhausted out of sealable volume 212. In such implementations, ozone destructor device 238 can, but need not, be activated at (314). For instance, as shown in FIG. 3, appliance 200 includes a venting conduit 240 that fluidly connects sealable volume 212 with a second volume, such as e.g., an ambient environment 244 or some other volume (e.g., another sealable volume of the appliance 200). A damper 242 movable between an open position and a closed position is positioned along venting conduit 240. When damper 242 is in the open position, fluid (e.g., air) is permitted to flow through venting conduit 240 (e.g., from sealable volume 212 to ambient environment 244). When damper 242 is in the closed position, fluid is prevented from flowing through venting conduit 240. Thus, when damper 242 is in the closed position, sealable volume 212 is in fact sealed.

Further, in some implementations, ozone O₃ can be forced or actively exhausted from sealable volume 212 through venting conduit 240. In such implementations, activating the one or more ozone removal devices at (314) includes activating an air handler. For instance, in such implementations, controller 220 can activate air handler 236 when damper 242 is moved the open position, e.g., to more rapidly move ozone O₃ from sealable volume 212. Controller 220 can activate air handler 236 and cause damper 242 to move to the open position simultaneously. Alternatively, the timing can be offset.

At (316), the method (300) includes once again measuring the concentration level of ozone within the sealable volume of the appliance. In some implementations, measuring the concentration level of ozone within the sealable volume includes receiving from a detection device, an input (e.g., a second input) indicative of the concentration level of ozone within the sealable volume and determining the concentration level of ozone within the sealable volume based at least in part on the received input (e.g., the received second input).

For instance, after controller 220 determines that the concentration level has reached the maximum concentration level threshold T_{MAX} at (310), controller 220 can receive, from ozone detection device 234, a second input indicative of a concentration level of ozone O₃ within the sealable volume 212. For example, controller 220 can receive one or more electrical signals indicative of the concentration level ozone O₃ within the sealable volume 212. Controller 220 can receive such signals, or the second input, and can determine the concentration level of ozone O₃ within sealable volume 212 based at least in part on the received second input. Accordingly, controller 220 measures the concentration level of ozone O₃ within sealable volume 212 of appliance 200 in much the same as done at (308).

At (318), the method (300) includes ascertaining whether the determined concentration level has reached a minimum concentration level threshold. For instance, based on the concentration level of ozone O₃ within sealable volume 212 determined at (314), controller 220 ascertains whether the determined concentration level has reached a minimum concentration level threshold T_{MIN}. The minimum concentration level threshold T_{MIN} can be set such that the concentration level is associated with a safe level for humans. For instance, the minimum concentration level threshold T_{MIN} can be set at a level that corresponds with an ozone concentration level that a consumer can safely open the door of the sealable volume 212.

For instance, as shown in FIG. 5, after the fifth dosage D5 is injected into sealable volume 212 by ozone generator 232, controller 220 ascertains at (310) that the concentration level of ozone O₃ within sealable volume 212 has reached the maximum concentration level threshold T_{MAX}, and accordingly, controller 220 ceases causing ozone generator 232 to inject predefined ozone dosages into sealable volume 212. Thereafter, at (318), controller 220 can ascertain whether the concentration level determined at (316) has reached the minimum concentration level threshold T_{MIN}. After reaching the maximum concentration level threshold T_{MAX}, the concentration level of ozone O₃ within sealable volume 212 decreases over time. As the concentration level of ozone O₃ decreases, controller 220 can monitor the concentration level, e.g., at (316), and can ascertain whether the concentration level has reached the minimum concentration level threshold T_{MIN}. Controller 220 can ascertain whether the concentration level has reached the minimum concentration level threshold T_{MIN} continuously or at a predetermined time interval. Eventually, as depicted in FIG. 5, the determined concentration level reaches the minimum concentration level threshold T_{MIN}. If the concentration level has reached the minimum concentration level threshold T_{MIN} (e.g., as shown in the first and second scenarios of FIGS. 5 and 6, respectively), then the method (300) proceeds to (324). If the concentration level has not reached the minimum concentration level threshold T_{MIN}, then method (300) proceeds to (320), and the logic remains in the (316), (318), and (320) loop until the concentration level reaches the minimum concentration level threshold T_{MIN} at (318) or a predetermined removal time elapses as determined at (320).

At (320), if the concentration level has not reached the minimum concentration level threshold T_{MIN}, then the method (300) includes determining whether a predetermined removal time has elapsed. For instance, controller 220 can maintain a timer or clock. The timer can be started when controller 220 ascertains at (310) that the concentration level determined at (308) has reached the maximum concentration level threshold T_{MAX} or at another suitable time, e.g., when the ozone destructor device 238 is activated at (314). If the predetermined removal time has not elapsed as determined at (320), then method (300) reverts to (316) and (318) to continue monitoring the concentration level. If, however, the predetermined removal time has elapsed as determined at (320), then the method (300) proceeds to (322).

In some implementations, particularly where appliance 200 includes ozone destructor device 238 and activates ozone destructor device 238 at (314), the predetermined removal time can correspond with a maximum destructor on time. In this way, ozone detector device 238 is prevented from running indefinitely in the event of a failure condition. In yet other implementations, particularly where appliance 200 includes venting conduit 240 and damper 242 and causes damper 242 to move to the open position to allow for ozone O₃ to exhaust out of sealable volume 212 through venting conduit 240, the predetermined removal time can correspond with a maximum exhaust time. In this manner, controller 220 need not attempt to exhaust ozone O₃ indefinitely, which may be of particular importance if sealable volume 212 is a chilled or otherwise conditioned chamber.

At (322), the method (300) includes detecting a fault condition and setting a fault condition flag associated with the detected fault condition. For instance, as shown in FIG. 4, the logic of method (300) can reach fault detection block (314) by multiple paths. For instance, in one path, if the concentration level determined at (310) does not reach the maximum concentration level threshold T_{MAX} before the maximum generator on time has elapsed as determined at (312), the method (300) proceeds to (322). In addition, in another path, if the predetermined removal time has elapsed at (320), the method (300) proceeds to (322). Accordingly, controller 220 first determines the fault condition and then sets a fault condition flag accordingly, or based at least in part on the detected fault condition.

As one example, if the concentration level determined at (310) does not reach the maximum concentration level threshold T_{MAX} before the maximum generator on time has elapsed as determined at (312), the detected fault condition can be at least one of 1) the ozone generator 232 has failed; 2) the ozone detection device 234 has failed; or 3) the sealable volume 212 is not sealed or air-tight, and accordingly, the injected ozone O₃ may be leaking from sealable volume 212. Based on the detected fault condition, controller 220 can set an associated fault condition flag.

As another example, if the predetermined removal time has elapsed at (320) and thus for some reason appliance 200 is unable to remove or reduce the ozone concentration level within sealable volume 212, the detected fault condition can be at least one of 1) the ozone destructor device 238 has failed (and thus the maximum destructor on time has elapsed at (320)); or 2) the damper 242 has failed or is clogged (and thus the maximum exhaust time has elapsed at (320)), among other possible fault conditions. Based on the detected fault condition, controller 220 can set an associated fault condition flag. Moreover, in some implementations, if the predetermined removal time has elapsed at (320), or more particularly, if the maximum destructor on time has elapsed at (320), the method (300) can further include deactivating ozone destructor device 238. Further, in some implementations, if the predetermined removal time has elapsed at (320), or more particularly, if the maximum exhaust time has elapsed at (320), the method (300) can further include deactivating air handler 236 and/or causing damper 242 to move to the closed position.

At (324), if the determined concentration level has reached the minimum concentration level threshold T_{MIN} as ascertained at (318), the method (300) includes deactivating one or more ozone removal devices. As one example, if ozone destructor device 238 is activated at (314) and the determined concentration level has reached the minimum concentration level threshold T_{MIN}, then deactivating the one or more ozone removal devices can include deactivating ozone destructor device 238. In this way, ozone destructor device 238 can be turned off. As another example, if ozone destructor device 238 is activated at (314) and the determined concentration level has reached the minimum concentration level threshold T_{MIN}, then deactivating the one or more ozone removal devices can include causing damper 242 to move to the closed position, e.g., to prevent air from escaping sealable volume 212 through exhaust conduit 240. As yet another example, deactivating the one or more ozone removal devices can include deactivating air handler 236.

At (326), the method (300) includes terminating the odor removal cycle. As shown the odor removal cycle can be terminated at (326) after deactivating the ozone devices at (324) or can be terminated after detecting a fault condition at (322). At the termination of the odor removal cycle, various information can be presented, e.g., to a user via a display of appliance 200. For instance, the degree or amount of odor, bacteria, viruses, and/or other contaminants within sealable volume 212 can be measured or calculated based on the amount of time it takes to remove them from sealable volume 212. For instance, the time can be measured from a start time to an end time. The start time can be associated with a time in which the first dosage of ozone is injected into sealable volume 212. The end time can be associated with a time in which the concentration level reaches the maximum concentration level threshold T_{MAX}. Other information can also be presented to the user as well.

In some implementations, the method (300) includes causing a door lock to lock a door of the appliance in the closed position during the odor removal cycle, e.g., from (302) to (326). For instance, controller 220 can cause, prior to causing ozone generator 232 to inject the predefined dosage of ozone O₃ into sealable volume 212 at (304), door lock 216 to lock door 214 in the closed position. Then, if controller 220 ascertains that the determined concentration level has reached the minimum concentration level threshold T_{MIN} at (318), controller 220 can cause door lock 216 to unlock such that door 214 can once again be opened. Accordingly, controller 220 can prevent a user from inadvertently interrupting the odor removal cycle and can protect a user from exposure to potentially unsafe levels of ozone O₃.

An appliance equipped with an ozone monitoring system and control logic of method (300) described herein can provide a number of advantages and benefits. For instance, the ozone monitoring system provided herein and implemented by the method can remove odor/bacteria from various appliances, including refrigerator, laundry, and air-conditioner appliances. Further, consumer safety is ensured by only injecting predefined amounts of ozone to remove odor/bacteria and can include a door lock mechanism to ensure consumers are not inadvertently exposed to unsafe levels of ozone. Moreover, consumers can execute an ozone removal cycle to remove odor/bacteria by using a small amount of energy without using heat energy.

This written description uses examples to disclose the invention, including the best mode, and also to enable any person skilled in the art to practice the invention, including making and using any devices or systems and performing any incorporated methods. The patentable scope of the invention is defined by the claims and may include other examples that occur to those skilled in the art. Such other examples are intended to be within the scope of the claims if they include structural elements that do not differ from the literal language of the claims or if they include equivalent structural elements with insubstantial differences from the literal language of the claims.

What is claimed is:

1. An appliance, comprising:
a housing defining a sealable volume;
an ozone generator operable to dispense ozone into the sealable volume;
an ozone detection device operable to detect a concentration level of ozone within the sealable volume;
a controller communicatively coupled with the ozone generator and the ozone detection device, the controller configured to:
i) cause, at a predetermined injection interval, the ozone generator to inject a predefined dosage of ozone into the sealable volume;
ii) receive, from the ozone detection device, an input indicative of the concentration level of ozone within the sealable volume;
iii) determine the concentration level of ozone within the sealable volume based at least in part on the received input; and
iv) ascertain whether the determined concentration level has reached a maximum concentration level threshold, and
wherein the controller iteratively i) causes, ii) receives, iii) determines, and iv) ascertains until the determined concentration level reaches the maximum concentration level threshold or a maximum generator on time has elapsed.

2. The appliance of claim 1, wherein, if the determined concentration level reaches the maximum concentration level threshold, the controller is further configured to:
receive, from the ozone detection device, a second input indicative of the concentration level of ozone within the sealable volume;
determine the concentration level of ozone within the sealable volume based at least in part on the received second input; and
ascertain whether the determined concentration level has reached a minimum concentration level threshold.

3. The appliance of claim 2, wherein, if the determined concentration level has not reached the minimum concentration level threshold within a predetermined removal time, the controller is further configured to:
detect a fault condition; and
set a fault condition flag associated with the detected fault condition.

4. The appliance of claim 1, wherein, if the determined concentration level does not reach the maximum concentration level threshold before the maximum generator on time has elapsed, the controller is further configured to:
detect a fault condition; and
set a fault condition flag associated with the detected fault condition.

5. The appliance of claim 1, further comprising:
an ozone destructor device operable to reduce the concentration level of ozone within the sealable volume, and
wherein, if the determined concentration level reaches the maximum concentration level threshold, the controller is further configured to:
activate the ozone destructor device to reduce the concentration level of ozone within the sealable volume;
receive, from the ozone detection device, a second input indicative of the concentration level of ozone within the sealable volume;
determine the concentration level of ozone within the sealable volume based at least in part on the received second input; and
ascertain whether the determined concentration level has reached a minimum concentration level threshold.

6. The appliance of claim 5, wherein if the determined concentration level has not reached the minimum concentration level threshold within a predetermined ozone destruction time, the controller is further configured to:
set a fault condition flag; and
deactivate the ozone destructor device.

7. The appliance of claim 1, further comprising:
a venting conduit fluidly connecting the sealable volume with a second volume;
a damper positioned along the venting conduit and movable between an open position and a closed position, wherein in the closed position, the damper prevents fluid flow through venting conduit, and wherein in the open position, the damper allows fluid flow through venting conduit, and
wherein, if the determined concentration level reaches the maximum concentration level threshold before the maximum generator on time has elapsed, the controller is further configured to:
cause the damper to move the open position;
receive, from the ozone detection device, a second input indicative of the concentration level of ozone within the sealable volume;
determine the concentration level of ozone within the sealable volume based at least in part on the received second input;
ascertain whether the determined concentration level has reached a minimum concentration level threshold; and
cause, if the determined concentration level has reached the minimum concentration level threshold, the damper to move to the closed position.

8. The appliance of claim 1, further comprising:
an air handler operable to move air within sealable volume;
wherein, if the determined concentration level reaches the maximum concentration level threshold before the maximum generator on time has elapsed, the controller is further configured to:
cause the air handler to move air within the sealable volume.

9. The appliance of claim 1, further comprising:
a door operatively coupled with the housing for providing selective access to the sealable volume, the door movable between a closed position in which the sealable volume is hermetically sealed and an open positon in which the sealable volume is not hermetically sealed; and
a door lock for selectively locking the door, the door lock communicatively coupled with the controller, and
wherein the controller is further configured to:
cause, prior to causing the ozone generator to inject the predefined dosage of ozone into the sealable volume, the door lock to lock the door in the closed position;
receive, from the ozone detection device, a second input indicative of the concentration level of ozone within the sealable volume;
determine the concentration level of ozone within the sealable volume based at least in part on the received second input;
ascertain whether the determined concentration level has reached a minimum concentration level threshold; and
cause, if the determined concentration level has reached the minimum concentration level threshold, the door lock to unlock the door.

10. The appliance of claim 1, wherein the appliance is one of a washing machine appliance, a dryer appliance, a dishwasher appliance, a microwave appliance, an oven appliance, and an air conditioner appliance.

11. The appliance of claim 1, wherein the appliance is a refrigerator appliance and the sealable volume is a chilled chamber of the refrigerator appliance.

12. A method for operating an appliance in an odor removal cycle, the method comprising:
injecting, at a predetermined injection interval, a predefined dosage of ozone into a sealable volume of the appliance;
measuring, after each injection of the predefined dosage of ozone into the sealable volume of the appliance, a concentration level of ozone within the sealable volume; and
ascertaining whether the concentration level has reached a maximum concentration level threshold, and wherein if the concentration level has reached the maximum concentration level threshold, then no further injections of the predefined dosage of ozone are made.

13. The method of claim 12, wherein if the concentration level of ozone within the sealable volume reaches the maximum concentration level threshold, the method further comprises:
measuring the concentration level of ozone within the sealable volume; and
ascertaining whether the concentration level has reached a minimum concentration level threshold within a predetermined removal time.

14. The method of claim 13, wherein, if the concentration level reaches the maximum concentration level threshold, measuring the concentration level of ozone within the sealable volume comprises:
receiving, from a detection device, a second input indicative of the concentration level of ozone within the sealable volume;
determining the concentration level of ozone within the sealable volume based at least in part on the received second input.

15. The method of claim 13, wherein, if the determined concentration level has not reached the minimum concentration level threshold within the predetermined removal time, the method further comprises:
detecting a fault condition; and
setting a fault condition flag associated with the detected fault condition.

16. The method of claim 12, wherein the predefined dosage of the ozone is injected into the sealable volume of the appliance at the predetermined injection interval by an ozone generator, and wherein if the concentration level has not reached the maximum concentration level threshold within a predetermined generator on time, the method further comprises:
detecting a fault condition; and
setting a fault condition flag associated with the detected fault condition.

17. The method of claim 12, wherein the appliance comprises a destructor device operable to reduce the concentration level of ozone within the sealable volume, and wherein, if the determined concentration level reaches the maximum concentration level threshold, the method further comprises:
activating the destructor device to reduce the concentration level of ozone within the sealable volume;
receiving, from a detection device, a second input indicative of the concentration level of ozone within the sealable volume;
determining the concentration level of ozone within the sealable volume based at least in part on the received second input; and
ascertaining whether the determined concentration level has reached a minimum concentration level threshold.

18. The method of claim 12, wherein the appliance comprises a venting conduit fluidly connecting the sealable volume with a second volume, the appliance further comprising a damper positioned along the venting conduit and movable between an open position and a closed position, wherein in the closed position, the damper prevents fluid flow through venting conduit, and wherein in the open position, the damper allows fluid flow through venting conduit, and
wherein, if the determined concentration level reaches the maximum concentration level threshold before the maximum generator on time has elapsed, the method further comprises:
causing the damper to move the open position;
receiving, an ozone detection device, a second input indicative of the concentration level of ozone within the sealable volume;
determining the concentration level of ozone within the sealable volume based at least in part on the received second input;
ascertain whether the determined concentration level has reached a minimum concentration level threshold; and
causing, if the determined concentration level has reached the minimum concentration level threshold, the damper to move to the closed position.

19. The method of claim 12, further comprising:
activating an air handler to facilitate diffusion of ozone within the sealable volume.
